(19) United States
(12) Patent Application Publication
(10) Pub. No.: US 2004/0235101 A1
(43) Pub. Date: Nov. 25, 2004

(54) Genetic circuit clocked latch

(76) Inventors: Kenzie D. Maclsaac, Cambridge, MA (US); Stephen W. Davies, Toronto (CA)

Correspondence Address: STEPHEN WILLIAM DAVIES, 4 Taddle Creek Rd., RS-407, TORONTO, ON, M5S 2S3, CA

(21) Appl. No.: 10/793,764

(22) Filed: Mar. 8, 2004

Related U.S. Application Data
(60) Provisional application No. 60/452,329, filed on Mar. 6, 2003.

(51) Int. Cl. C07K 14/415; C07H 21/04; A01H 1/00; C12N 15/82

(52) U.S. Cl. 435/69.1; 800/278; 435/252.33; 435/320.1; 530/350; 536/23.5

(57) ABSTRACT

We describe methods and compositions for setting and maintaining the state of gene expression in a cell. The genetic circuit clocked latch has two states with each state corresponding to a different pattern of gene expression. The genetic circuit clocked latch allows one to set the state of the latch only when the clock signal is active. When the clock signal is inactive, the state of the latch is maintained as the state when the clock signal was last active. The clocked latch consists of gating circuitry and a bistable switch. The circuit is implemented as nucleic acid constructs. By analogy with a similar circuit found in digital electronics, such a circuit is expected to have an increased immunity to input fluctuations and is expected to facilitate the design of large circuits composed of multiple latches.

### CROSS-REFERENCE TO RELATED APPLICATIONS

This application claims priority to, and the benefit of U.S. Ser. No. 60/452,329, filed on Mar. 6, 2003, the disclosures of which are incorporated by reference herein.

### STATEMENT REGARDING FEDERALLY SPONSORED RESEARCH

United States Federal sponsorship was not involved in this work.

### REFERENCE TO A MICROFICHE APPENDIX

Not applicable.

### BACKGROUND OF THE INVENTION

### REFERENCES

1. Becskei, A., Serrano, L., “Engineering stability in gene networks by autoregulation”, Nature 405, 1 Jun. 2000, pp.590-593.

2. McAdams, H. H., Arkin, A., “Gene regulation: towards a circuit engineering discipline”, Current Biology, Vol. 10, No. 8, 2000, pp.R318-R320.

3. Kennell, D., Riezman, H., “Transcription and translation initiation frequencies of the Escherichia coli lac operon”, J. Mol. Biol., Vol.114, 1977, pp.1-21.

4. McClure, W. R., “Rate-limiting steps in RNA chain initiation”, Proc.Natl.Acad.Sci.USA, Vol.77, No.10, Oct., 1980, pp.5634-5638.

5. Yarchuk, O., Jacques, N., Guillerez, J., Dreyfus, M., “Interdependence of translation, transcription and mRNA degradation in the lacZ gene”, J.Mol.Biol., Vol.226, 1992, pp.581-596.

6. Lombardi, L., Ciana, P., Cappellini, C., Trecca, D., Guerrini, L., Migliazza, A., Maiolo, A. T., Neri, A., “Structural and functional characterization of the promoter regions of the NFKB2 gene”, Nucleic Acids Research, Vol.23, No.12, 1995, pp2328-2336.

7. Keller, A. D., “Model genetic circuits encoding autoregulatory transcription factors”, J.theor.Biol., Vol.172, 1995, pp.169-185.

8. McAdams, H. H., Arkin, A., “Stochastic mechanisms in gene expression”, Proc.Natl.Acad.Sci. USA, Vol.94, Feb., 1997, pp.814-819.

9. Little, J. W., Shepley, D. P., Wert, D. W., “Robustness of a gene regulatory circuit”, EMBO J., Vol.18, No.15, 1999, pp.4299-4307.

10. Gardner, T. S., Cantor, C. R., Collins, J. J., “Construction of a genetic toggle switch in Escherichia coli”, Nature, Vol. 403, 20 Jan. 2000, pp.339-342.

11. Elowitz, M. B., “A synthetic oscillatory network of transcriptional regulators”, Nature, Vol.403, 20 Jan. 2000, pp.335-338.

12. Maloy, S. R., Cronan, J. E., Freifelder, D., “Microbial genetics”, Jones and Bartlett, Sudbury, Mass., USA, 1994, pp.131-133.

13. Sambrook, J., Russell, D. W., “Molecular Cloning: A Laboratory Manual, 3^{rd} ed. Cold Spring Harbour Laboratory Press, Cold Spring Harbour, N.Y., 2001.

14. Lutz, R., and Bujard, H., “Independent and tight regulation of transcriptional units in Escherichia coli via LacR/O, the TetR/O and AraC/I1-12 regulatory elements”, Nucleic Acids Research Vol. 25, 1997, pp. 1203-1210.

15. “Tools and technology for life sciences”, Stratagene Corp., LaJolla, Calif., USA, 2001, pp.40-41.

16. De Rubertis, G., Davies, S. W., “Genetic circuit design from an electronics perspective”, IEEE Conf. Molecular, Cellular and Tissue Engineering, Genoa, Italy, 6-9 Jun. 2002, pp. 123-124.

17. De Rubertis, G., Davies, S. W., “Design and simulation of a genetic circuit amplifier”, 10th Intl. Conf. on Intelligent Systems for Molecular Biology, Edmonton, Canada, Aug. 3-7, 2002, pg.113.

18. DeRubertis, G., M.A.Sc. thesis, University of Toronto, 2002.

19. Seale, D., Davies, S. W., “Stochastic model of DNA microarrays”, IEEE Conf. Molecular, Cellular and Tissue Engineering, Genoa, Italy, 6-9 Jun. 2002, pp.113-114.

20. Seale, D., Davies, S. W. “Performance analysis of an optimal estimator of gene expression ratio”, 10th Intl. Conf. on Intelligent Systems for Molecular Biology, Edmonton, Canada, Aug. 3-7, 2002, pg.55.

21. Seale, D., Davies, S. W., “Optimal estimation of gene expression”, IEEE 2nd Joint EMBS-BMES Conf., Houston, USA, 23-26 Oct. 2002.

22. MacIsaac, K., Davies, S. W., “Maximum likelihood estimation of rate parameters in single-molecule observable biological systems”, 27th Canadian Medical and Biological Engineering Conference, Ottawa, Ontario, Nov. 21-23, 2002.

23. Brown, S., Vranesic, Z., “Fundamentals of digital logic with VHDL design”, McGraw-Hill, Boston, 2000, pp. 338-340.

24. Ptashne, M., “A genetic switch”, 2ed., Blackwell Science Inc., Malden, Mass., USA, 1992.

25. Kay, M. A., Glorioso, J. C., Naldini, L., “Viral vectors for gene therapy: the art of turning infectious agents into vehicles of therapeutics”, Nature Medicine, Vol. 7, No. 1 Jan. 2001, pp.33-40.

26. Clarkson, T., “Regulated gene expression systems”, Gene Ther., Vol. 7, 2000, pp.120-125.

27. Winter, P. C., Hickey, G. I., Fletcher, H. L., “Genetics”, 2^{nd} Ed., BIOS Scientific Publishers, Oxford, UK, 2002, pp.57-58.

28. Wagner, R., “Transcription Regulation in Prokaryotes”, Oxford University Press, Oxford, UK, 2000.

29. Yokbayashi, Y., Weiss, R., Arnold, F. H., “Directed evolution of a genetic circuit”, PNAS early edition, www.pnas.org/cgi/doi/10.1073/pnas.252535999, 2002, pp.1-5.

30. Leung, D. E., Chen, E., Goeddel, D. V., “A method for random mutagenesis of a defined DNA segment using a modified polymerase chain reaction”, Technique, Vol. 1, pp.11-15.

31. Shimizu-Sato, S., Huq, E., Tepperman, J. M., Quail, P. H.,“A light-switchable gene promoter system”, Nature Biotech., Vol. 20, No. 10, 2002, pp.1041-1044.

32. Weiss, R., “Cellular Computation and Communications using Engineered Genetic Regulatory Networks”, Ph.D. Thesis, M.I.T., Boston, August 2001

33. Yao, F., Eriksson, E., “A novel tetracycline-inducible viral replication switch”, Human gene therapy, Vol. 10, No. 3,Feb. 10, 1999, pp. 419-27.

34. Baron, U., Gossen, M., Bujard, H., “Transcriptional activators with graded transactivation potential”, U.S. Pat. No. 6,271,341, Aug. 7, 2001.

35. Arkin, A. P., “Signal processing by biochemical reaction networks”, in Self Organized Biological Dynamics & Nonlinear Control, Walleczek, J., Editor,Cambridge University Press, Cambridge, UK, 2000,pp. 112-144.

36. Kim, D. H., McCormick, F., “Replicating viruses as selective cancer therapeutics”, Mol. Med. Today, Vol. 2, 1996, pp.519-527.

37. Ramachandra, M., et al, “Re-engineering adenovirus regulatory pathways to enhance oncolytic specificity and efficacy”, Nature Biotech., Vol. 19, No. 11, 2001, pp.1035-1041

38. U.S. patent application 30166191, Gardiner, T., et al, Sep. 4, 2003.

A genetic circuit is an intriguing way to view the signaling and feedback pathways in a cell. Here, the signaling molecules are seen as the wires in the circuit, connecting each output to another input. The phrase “genetic circuit” actually dates from the early 1960's but it has acquired new vitality as we enter the infancy of genetic circuit engineering [2]. In the intervening period, existing circuits in living creatures were deduced. Researchers measured and characterized the behaviors (e.g. binding constants) of components of genetic circuits [3-6]. Theoretical models of regulatory genetic circuits were developed [7-9]; in particular, these models addressed the molecule-by-molecule stochastic nature of gene control circuits. In the same period, the tools of genetic engineering, such as cloning [13], were developed. Recently, synthetic genetic circuits have been created that demonstrate a number of basic engineering functions. A toggle switch, a regulator and an oscillator have been reported.

The “toggle switch” is a digital genetic circuit with outputs being either on or off[10,38]. This device enters the “on” state when a certain input signal (isopropyl-β-D-thiogalactopyranoside (IPTG)) is applied and remains in that state when the input is removed. Applying a different kind of input signal (anhydrotetracycline(aTc) or heat, depending on the promoter used) sends it to the “off” state where it remains when said input is removed. The IPTG inducer interacted with lacI repressor and promoter Ptrc-2, all basically from the lactose operon, the operon explained first in microbial genetics texts. The aTc inducer interacted with repressor tetR and promoter P_{L}tetO- 1. A gene for green fluorescent protein (gfp) was used to produce the output signal. The plasmid was inserted into an E. coli strain.

The regulator, an analog genetic circuit, is analogous to the voltage or current regulator found in electronic circuits. The investigation centered on the variation of the preset output level in the face of external disturbances [1]. The plasmid implementation centered on the commonplace repressor (tetR) for tetracycline resistance and its corresponding operator sequence. A gene for green fluorescent protein (gfp) was used to produce the output signal. The plasmid was inserted into an E. coli strain.

Using similar components, an oscillator has been created [11]. This “Repressilator” features three promoters, for CI, TetR and LacI, connected in a circle. An active promoter 1 turns off the promoter 2 at its output which in turn allows the promoter 3 following that one to turn on and suppress promoter 1. Thus a wave of activity cycles through the promoters in reverse order.

Commercial sources have advanced to where they are now supplying two plasmid systems for studying interactions between two proteins. Plasmids providing intracellular protein output proportional to a membrane diffusable agent (e.g. IPTG) are also available [15].

Both analog and digital functions are under investigation in the applicants' laboratory. In addition to the instant invention, a genetic circuit-based amplifier has been designed, built and tested [16-18]. In standard instrumentation, analog and digital electronics are integrated together to provide an instrumentation system. Our work has applied optimal signal processing techniques to molecular biological instrumentation problems[19-22]. In general, our work in genetic circuits seeks to integrate analog and digital functional blocks to provide a living instrumentation system.

### FIELD OF THE INVENTION

The present invention relates generally to methods and compositions for regulating gene expression in a cell. In particular, the invention relates to genetic switch constructs that can switch the expression of one or more genes between a stable “on” state and a stable “off” state or vice versa in response to a transient stimulus.

### BRIEF SUMMARY OF THE INVENTION

This invention concerns implementation of what is known as a genetic circuit, which is a set of genes engineered to perform one or more signal processing functions, much like electronic circuits do. The instant invention is of a memory element, a clocked latch. This element maintains a particular logical state until a clock signal becomes active. When the clock is active the logical state is determined by the current input.

Synthetic genetic circuits have been created that demonstrate a number of basic engineering functions. A toggle switch, a regulator and an oscillator have been reported. The instant circuit features a bistable switch, similar to the toggle switch, to provide basic information storage. The key innovation is to add gating circuitry that restricts the inputs to influence the output only while the clock is in a particular state.

As proven in digital electronic circuits, clocked memory elements offer several advantages over non-clocked systems including increased resistance to noise, reduced susceptibility to undesirable “race” conditions, and increased ease of design of large systems. Programmed intracellular genetic circuits based on this technology may aid investigations into cell differentiation and cancer.

### BRIEF DESCRIPTION OF THE DRAWINGS

FIG. 1 is a schematic representation of a D-latch.

FIG. 2 is a generic representation of a genetic circuit clocked latch.

FIG. 3 is a schematic representation of a preferred genetic circuit clocked latch illustrating the specific genes, promoters and control elements used as well as their relative positions on the construct.

FIG. 4 is a schematic representation of the plasmid incorporating the preferred genetic clocked latch.

### DETAILED DESCRIPTION OF THE INVENTION

The instant invention is of a memory element, a clocked latch. This genetic circuit element, inspired by digital electronic D-latch, maintains a particular logical state until a clock signal becomes active. When the clock is active the logical state is determined by the current input. The instant circuit features a bistable switch to provide basic information storage. The key innovation is to add gating circuitry that restricts the inputs to influence the output only while the clock is high.

### 1. D-Latch Function and Structure

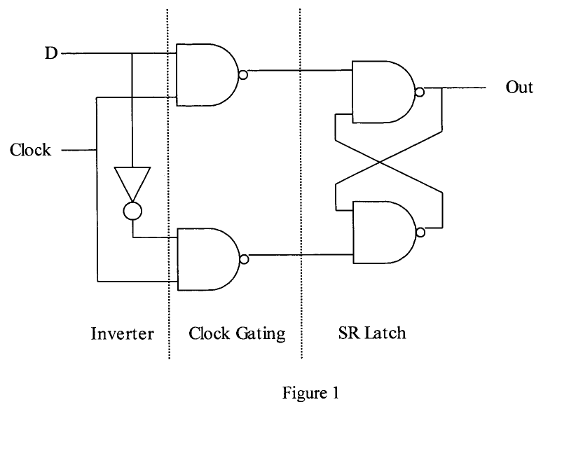

FIG. 1 shows a typical digital logic circuit implementation of a D-latch [23]. There are three main sections to this particular implementation. The cross-coupled gates to the right of the figure provide basic set-reset (SR) latch capability that allows for the preservation of the data value. This element is analogous to the genetic circuit toggle switch mentioned earlier. To the left of the SR-latch is a pair of gates that allow the clock to control the application of set and reset values to to SR-latch. Finally, the third section is comprised of the single inverter which allows set and reset values to be created form the input data, D.

These gates are NAND gates wherein their output can only be logic zero if both their inputs are logic one. Otherwise their output is logic one. Thus when one gate of the SR-latch has a logic zero output then it forces the other to be logic one, regardless of the other's input. The first gate will maintain its logic zero output as long as its other input is logic one.

The clock gating circuitry has both outputs logic one when the clock is logic zero. Thus the inputs to the SR-latch will be logic one and will not affect the latch output value. When the clock goes to logic one then the not S output will be logic zero if the data input D is logic one and the not R output will be logic zero if the data input D is logic zero. Whichever is logic zero will force the latch into the state that matches the value of D while the clock is high. The input output relations for the D-latch are described in Table 1.

TABLE 1
Input output relation for a D-latch. Here t denotes the time of the
last logic one clock pulse and t + 1 denotes the output at the
end of the current logic one clock pulse.

| Clock | Input Data, D | Output, Q(t + 1) |
|---|---|---|
| 0 | Don't care | Q(t) |
| 1 | 0 | 0 |
| 1 | 1 | 1 |

Many variants of this design are possible. One design replaces the NAND gates with NOR gates (the output of a NOR gate is zero except if both inputs are zero). Another replaces the SR-latch with another feedback arrangement. Common to all designs is a means of controlling by a clock signal the progression of the input information to the storage element.

Also, it must be noted that other basic clocked circuit components are easily formed from the combination of gating circuitry and a bistable switch. These include a gated SR-latch (i.e. the above circuit without the inverter), master-slave D flip-flop (two D-latches in cascade with the second driven by an inverted clock signal), T flip-flop (D flip-flop based clocked toggle switch) and JK flip-flop (D flip flop incorporating both SR latch and toggle modes) [23]. Thus demonstration of D-latch capability indicates the capability to demonstrate these other circuits as well. Similarly, registers, multi-bit parallel structures of D-latches, are also implicitly demonstrated.

The gated SR-latch is a good low complexity demonstrator of a clocked memory element. With the trivial addition of an inverter such as described by Weiss [32], it can be made into a D-latch. Thus demonstration of an SR-latch demonstrates the capability to create the diverse range of clocked circuits described above. The clocked latch implementation to be described herein is equivalent to a gated SR-latch.

### 2. Gating Transcription Signals

Clocked latches are typically used in complex systems composed of many latches. The most common arrangement features all latches controlled by the same clock. This allows information to progress through the circuit from latch to latch in a systematic and predictable manner. Systems without such a common clock, known as asynchronous systems, present daunting challenges to the system designer. Thus, it is desirable that the selected gating arrangement for genetic circuits permit the common clock functionality.

Transcription is a complex process in both prokaryotic and eukaryotic cells. There are a variety of means to modulate it. The most basic mechanism in prokaryotes is the inducer-repressor operator promoter paradigm. A repressor molecule, usually the product associated with the gene driven by the promoter, will bind specifically to the promoter region and turn off transcription. To turn on transcription, a similarly specific inducer molecule can bind to the repressor, freeing the promoter for gene expression.

The key term to note is specific. The inducer-repressor-promoter combination is very specific. As genetic circuit designers, we would like to exploit that specificity to achieve complex circuits with many specific interconnections.

An effective mechanism for clocking these circuits would permit such complex interconnections on the data inputs and outputs but have a simple common mechanism, at the molecular level, for clocking the circuits.

Secondary structure in DNA and RNA provides a potential mechanism. DNA and RNA have the property that complementary sequences in single strands can bind to one another to form stem and loop structures. These secondary structures can interfere with the progression of polymerases and/or ribosomes along the DNA or RNA.

A temperature sensitive stem and loop structure could be engineered into the mRNA transcript. At low temperature, the engineered stem and loop in the RNA prevents translation and hence output and feedback. High temperature releases the bonds, permits RNA translation and therefore allows the inputs to affect output and feedback as in [10]. For a design based on this mechanism, the clock signal would be a periodically cycling culture temperature. The stem and loop structure would be placed between the specific input promoters and their associated genes.

Alternatively, secondary structure can lead to the RNA polymerase separating from the template and aborting the transcript. Transcriptional termination occurs when RNA polymerase transcribes through a DNA terminator sequence, producing RNA with specific three-dimensional structure that induces a pause in transcription. Whether transcription continues is determined kinetically by the relative rates of the RNA polymerase dissociation reaction and the elongation reaction for the paused complex. Termination frequencies are known to be as high as 95% for certain terminator sequences.

The most elementary example of this form of transcription regulation occurs in the synthesis of tryptophan. The attenuator in the trp operon [12] effects this regulation by forming alternative stem and loop structures depending on the concentration of tryptophan. The attenuator sequence incorporates the code for a pair of tryptophan molecules. In the case of an excess of tryptophan, the ribosome quickly incorporates the appropriate tRNA's and moves past this section. A special stem and loop structure, a terminator, forms and transcription is terminated. However, in the case of low tryptophan, the ribosome stalls at the double tryptophan codons. This allows a gap to form between the progressing RNA polymerase and the stalled ribosome. Within the gap a stretch of RNA is exposed which forms an alternative stem and loop. A portion of the RNA that would have participated in the formation of the terminator structure is now in the alternative stem and loop, so the terminator cannot form. This allows transcription to continue past the terminator sequence. The alternative stem and loop is referred to as the anti-terminator. A design based on this mechanism would use a periodically cycling level of a tRNA/amino acid as the clock signal.

More generally, terminators and anti-terminators are used to regulate other intracellular molecules. Antitermination is an important regulatory mechanism in vivo. In transcriptional antitermination, a protein factor, or a group of factors, form a complex with a transcribing RNA polymerase that makes it resistant to pausing at termination sites. These termination sites can in the simplest case be viewed as open switches that prevent transcription of genes located downstream. The switch is closed only when the required antitermination factors are present in the cell. The switch is reopened when these termination factors are degraded or diluted sufficiently through cell growth and division.

For example, in phage lambda, there is a DNA sequence consisting of the N utilization (NUT) site, an arbitrary spacer sequence and a rho-independent terminator structure known as t_{L3}. In the absence of N protein, transcription terminates at t_{L3}. However, in the presence of N protein, the transcribing assembly can form a complex with the N protein. This complex is resistant to termination at the t_{L3} site.

### 3. Memory in Genetic Circuits

The core of any digital memory element is a bistable switch, a circuit that can remain in either one of two stable states. The synthetic genetic circuit toggle switch is an example of a bistable switch. There are many naturally occurring bistable genetic switches. A classic example of a naturally occurring bistable genetic switch selects between the lysogenous and lytic phases in bacteriophage lamda [24].

As described in the section on D-latch function and structure, a bistable switch can be formed through cross-coupled feedback wherein being in one state actively locks out the other state. An alternative view would have a bistable circuit emerging from any circuit with positive feedback wherein the input can drive it to one of two stable states. There are clearly a wide variety of structures that can lead to bistability. They vary with respect to complexity.

### 4. Genetic Circuit Clocked Latch

The instant invention consist of clock gating circuitry and a bistable switch. The clock gating circuitry employs a stem and loop mechanism to control transcription/translation.

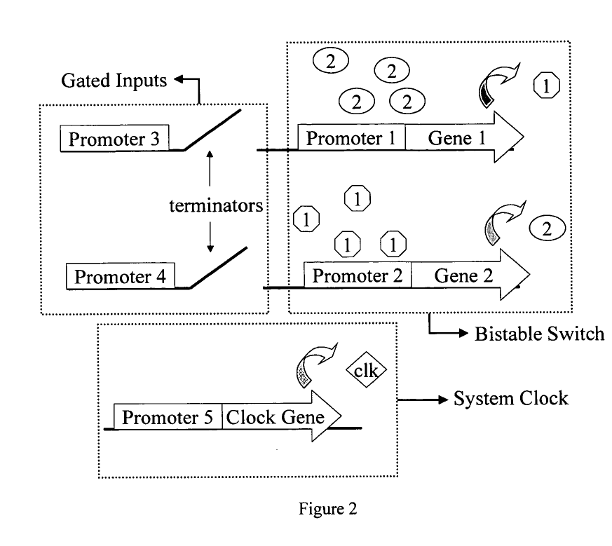

A schematic diagram of the genetic latch design is shown in FIG. 2. The bistable switch incorporates the co-repressing proteins encoded by gene 1 and gene 2. The gene 1 product binds to promoter 2 and prevents transcription of gene 2. The gene 2 product binds to promoter 1 and prevents transcription of gene 1. Switching is accomplished by driving the switch to one of these two states using promoters 3 and 4. These promoters can be controlled via some external input to the cell, for example a specific growth media, temperature, or chemical signal. Alternatively they could be connected to the output of another genetic circuit to form a larger network.

In practice only one driving input is active at a time since one would typically not attempt to drive the switch to the on and off states simultaneously. With this limitation, the circuit in FIG. 2 may be interpreted as a D-latch. More precisely, given the two inputs, the circuit is more properly denoted as a gated SR-latch. By adding a constitutive promoter that is repressed by the input to promoter 3, and having it make the input for promoter 4, an inverter is provided to form a proper D-latch.

These inputs are gated by the terminators. The terminators are represented in FIG. 2 as open switches. These can prevent transcription through to genes 1 and 2.

The clock gene, controlled by promoter 5, codes for an antiterminator protein. This protein will allow transcription to proceed through the terminators. Alternative designs could feature different terminators and their corresponding clock sources (e.g. tRNA or temperature). These alternative terminators would replace the terminators of FIG. 2.

When the clock signal is on then the input signals controlling promoters 3 and 4 can pass through and drive the switch. When the clock signal is off, then the bistable switch maintains its current state regardless of the signals driving promoters 3 and 4.

### 5. Preferred Implementation

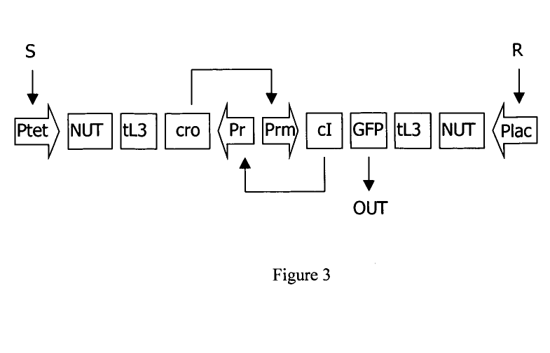

FIG. 3 is a schematic representation of the preferred implementation.

The bistable switch component of this design consists of the P_{RM} and P_{R} promoters from phage lambda assuming the roles of promoters 1 and 2 respectively in FIG. 2. The cI and cro genes of phage lambda correspond to genes 1 and 2. As depicted in FIG. 3, when P_{RM} is active, cI is transcribed and CI binds to P_{R} to shut off transcription of cro. Similarly the reverse holds when P_{R} is active and cro suppresses transcription of cI.

The clock gating circuitry consists of an N utilization (NUT) site in tandem with a rho-independent terminator structure known as t_{L3}. These were also obtained from phage lambda.

The promoters Pₜₑₜ and P_{lac} in FIG. 3 correspond to promoters 3 and 4, respectively, of FIG. 2. Here S and R indicate the input molecules that activate Pₜₑₜ and P_{lac}, respectively. The set (S) input, through activation of Pₜₑₜ can lead to expression of GFP; the reset (R) input can turn GFP off. Assuming the clock is active, when Pₜₑₜ is active, an mRNA transcript stretching from Pₜₑₜ to GFP is made. This leads to the expression of CI and GFP. This shuts off transcription of cro and locks the bistable switch in the P_{RM} active state.

On the other hand, if the clock was active and P_{lac} was active, an mRNA transcript stretching from P_{lac} to cro is made. This leads to the expression of cro and locks the bistable switch in the other state.

When the clock is inactive, the t_{L3} sites lead to termination and thus prevent either Pₜₑₜ or P_{lac} from affecting the state of the bistable switch.

Note that the active clock state corresponds to a high concentration of N antiterminator protein. A separate genetic circuit is used to make this clock signal.

### 6. Fabrication

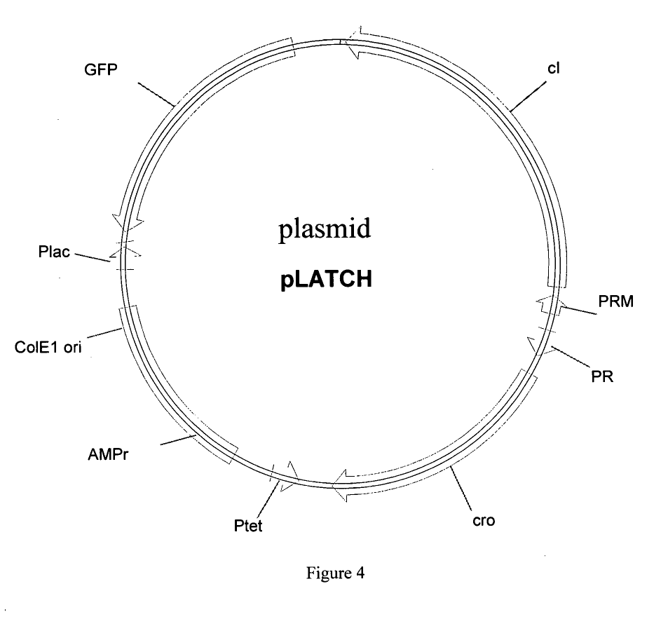

The pLatch plasmid depicted in FIG. 4 contains the bistable switch and clock gating circuitry. The pLatch plasmid was fabricated through several rounds of insertion and transformation. The sequence of plasmid fabrications was pSwitch2, pSwitch3, pSwitch4 and finally pLatch. The first plasmid, pSwitch2, contains the bistable switch components and half of the gating circuitry. The next, pSwitch3, adds a GFP reporter to serve as the output signal. The other half of the gating circuitry is added in pSwitch4. Input promoters were then added to pSwitch4 to create pLatch. The clock signal is generated using another plasmid not shown in FIG. 4. The clock plasmid, pClock, was made in a single insertion and transformation. The entire construction process is detailed in the remaining paragraphs of this section.

The DNA region containing the P_{R}/P_{RM} operator, the cI gene, and the cro gene of bacteriophage lambda was obtained via PCR amplification from bacteriophage lambda DNA as described in [13]. BamHI and EcoRi restriction endonuclease sites were introduced to the PCR product via the SWITCHfwd and SWITCHrev primers (sequences provided in Appendix A). The t_{L3} terminator and N-utilization site from bacteriophage lambda, flanked by EcoRi and XhoI cohesive ends, was obtained by total synthesis (TermPos and TermNeg; sequences provided in Appendix A). The pSwitch2 plasmid was obtained by a three-way ligation of these two DNA fragments into the pBluescript II SK(+) plasmid. This was then used to transform chemically competent DH5α cells via heat shock as described in [13].

The GFPaav gene was PCR amplified from the repressilator plasmid [12]. BamHIl and NotI restriction sites were introduced to the PCR product via the GFPfwd and GFPrev primers (sequences provided in Appendix A). A point mutation was introduced into the GFPrev primer to obtain the GFPaav variant from the GFPasv template. The pSwitch3 plasmid was obtained by cloning the digested GFPaav PCR product into the pSwitch2 plasmid using the protocol described in [13]. Chemically competent DH5α cells were transformed with pSwitch3 as described in [13].

A DNA fragment containing the t_{L3} terminator and N-utilization sites of bacteriophage lambda was obtained by total oligonucleotide synthesis (Term2Pos and Term2Neg; sequence provided in appendix A). It included flanking SacI and NotI cohesive ends. This DNA fragment was cloned into the pSwitch4 plasmid using protocol [13]. Chemically competent DH5α cells were transformed with pSwitch4 as described in protocol [13].

The pSwitch4 insert, consisting of the cI/cro switch region, the GFPaav reporter, and the flanking NUT/tL3 terminator pairs, was amplified by PCR from the pSwitch4 template using the LATCHfwd and LATCHrev primers (sequences provided in appendix A). The LATCHfwd primer introduced a flanking ClaI restriction endonuclease site and the P_{LtetO-1} promoter region (Bujard). The LATCHrev primer introduced a flanking XmaI restriction endonuclease site and the P_{LlacO-1} promoter region (Bujard). The PCR product was digested with ClaI and XmaI, and was cloned into a pBT plasmid (Stratagene Corporation) according to the standard protocol described in [13]. This yielded the pLATCH plasmid which was then used to transform chemically competent DH5αZ1 E. coli as described in [13].

To construct the clock plasmid, the N gene from bacteriophage lambda, encoding the antitermination factor N, was first amplified by PCR. The Nfwd and Nrev primers incorporated flanking EcoRI and BamHI sites into the PCR product (sequences provided in Appendix A). The N gene was then cloned into the pBAD expression vector (courtesy Beckwith laboratory, Harvard University) using standard protocols described in [13]. The resulting pClock plasmid was used to transform chemically competent DH5αZ1 E. coli containing the pLatch plasmid, as described in [13].

### 7. Implementation Variations

In addition to the preferred implementation(s) discussed earlier, and simple intuitive variations, the following types of variations on these implementations are explicitly included in this application:

First, variation is possible through substitution of alternative operators, promoters and genes from bacterial, viral and eukaryotic sources. Active proteins, transcription factors, repressors, inducers, enhancers, ligands, reporters and silencers [27] should then be selected to function with these alternatives. Logically and functionally, the substituted units behave the same as those originally specified; however, the active molecules carrying out these functions are different.

Second, variation is possible through the use of any desired or necessary (if required for a specific organism) transformation vector or vectors to carry the circuit or circuits into the organism. These vectors may then effect replication of the circuit(s) if so desired. They may also include, either directly or genetically, additional active machinery such as polymerases and ribosomes to effect or enhance circuit activity.

Third, variation is possible through changing the location of individual operator, promoter and gene structures on the vector or vectors. For example, a circuit composed of two different operons (i.e. promoters and genes) may have both operons placed on the same plasmid or, alternatively, such a circuit may have each of the two operons on a different but compatible plasmid. Further, when certain circuit molecules are capable of logically linking different cells, through for example ligand-receptor interactions or diffusible hormones, then different elements of the circuit may reside in different cells and if necessary, different vectors may be used to effect the respective transformations.

Fourth, variation is possible through the use of components naturally present in the host cell to form part of the circuit. This hybrid circuit is formed of host and vectored components.

### 8. Coupling Internal Circuit Signaling to Desired Cellular or Environmental Signals

The genetic circuits described in this application will typically use molecular species not naturally occurring in the host cell for internal signaling within the genetic circuit itself. This is to minimize potentially harmful unintentional interactions between circuit and cell. However, to effect a desired impact on cellular activities, circuit inputs and/or outputs will need to be coupled to host pathways.

The easiest means to do this is to use elements that naturally couple to host signals. For example, the circuit output may be coupled to the host via having the circuit output promoter coupled to a gene for the host pathway molecule at the desired point of connection. For example, the circuit input may respond to the host via including as the input promoter one whose operator is affected by a repressor, activator, inducer, enhancer, silencer or other transcription factor that is made by the host at the desired point of coupling host output to circuit input. More elaborate is a specially engineered or evolved coupling molecule or coupling molecule complex.

External input to the circuit may be through diffusible molecules based on metabolic substrates such as IPTG, natural diffusible signals such as hormones, or ligand-receptor interaction. Mechanical or electromagnetic (e.g. light [31]) inputs may also be coupled to circuit input via molecular transduction. External output from the circuit may be via a diffusible molecule or a diffusible complex of molecules as in a viral capsid. Output may be through reporters based on fluorescence, chromatographic (e.g. color) change or binding to detectable tag molecules. Transport of molecules in or out of the cell and/or in and out of the nucleus may be through passive diffusion or facilitated by transporter molecules. Access to circuit state as recorded on a nucleic acid, peptide or other non-diffusible molecule may be through destructive lysis of the cell and extraction and identification of signal molecule(s).

### 9. Genetic Circuit Hosts

### a) Prokaryotes

The prior art synthetic genetic circuits were constructed using plasmids as transformation vectors. Promoters and attendant operators used as circuit components were drawn from bacteriophages (bacterial viruses). These vectors and components may be used to construct the circuits presented herein.

In addition, a range of other standard transformation vectors may be used including bacteriophages, cosmids and phagemids to carry the circuit into the host bacterium. Promoters and operators used in circuit construction may also be drawn from other bacterial species. In anticipation of future developments, entirely new synthetic promoters may be created as per research underway by Professor M. Surette at the University of Calgary. Alternatively, genetic regulatory elements from higher organisms may be used provided that the cells may be augmented with additional RNA polymerases and/or ribosomes that can recognize the corresponding transcriptional and translational control sequences. Tuning of circuit interactions (such as an adjustment of reaction rate constants as in repressor operator binding for example) may be accomplished through mutation of corresponding DNA sequences and selection of mutations with desired properties [29-30].

### b) Eukaryotes

Standard transfection vectors may be used to insert the circuit(s) into eukaryotic cells. Standard laboratory transfection vectors may be used. Vectors used in gene therapy [25] such as those based on any of basic retroviruses, lentiviruses, adenoviruses or herpes simplex viruses may be used to introduce the genetic circuit into mammalian cells. Transformation vectors in use for plants may be used to add synthetic genetic circuits to plants. Yeast transformation vectors will allow addition of these circuits to yeast.

For eukaryotic cells, the promoters and transcription factors (including enhancers and silencers as analogs to the activators and repressors of bacterial promoters) that serve as genetic circuit components may come from eukaryotic cells or viruses that infect eukaryotic cells. Typically, the components will have viral sources [26]. For example, Yao [33] has created a controllable promoter structure for eukaryotes by taking the cytomegalovirus (CMV) promoter and adding bacterial tetracycline operator sequences just downstream from it. The result is an expression system that may express any desired gene in eukaryotes with expression level modulated by the tetracycline repressor molecule. Bujard's lab [34] have created controllable expression systems for eukaryotes by creating fusion proteins consisting of a protein that will bind to a DNA sequence of interest and the protein VP 16 transcriptional activation domain of Herpes Simplex Virus 1. Introduction of this protein leads to the expression of the gene placed after the sequence of interest. Additionally, it is anticipated that new synthetic promoters and transcription factors capable of functioning in eukaryotic cells will be available in the near future. Tuning of circuit interactions, an adjustment of reaction rate constants as in transcription factor binding for example, may be accomplished through mutation of corresponding DNA sequences and selection of mutations with desired properties [29-30]. Less likely sources of components are bacterial or bacteriophage promoters; these will require the introduction into the cells of machinery (polymerases and ribosomes) capable of interpreting the corresponding transcription and translation signal sequences.

Finally, for eukaryotic systems, the circuits may exploit systems for active transport into (and/or out of) the nucleus as part of the circuit itself. Another variation would use mitosis for access to chromatin; this could be for transformation or for synchronizing circuit state with mitosis.

### 10. Notes

Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are intended to be embraced therein.

Incorporation by Reference

Each of the patent documents and scientific publications disclosed herein is incorporated by reference into this application in its entirety.

What is claimed is:

1. A recombinant genetic clocked latch comprising:
(a) a bistable genetic switch that is capable of being substantially stable in a first state or in a second state, the switch comprising nucleic acid constructs, each construct comprising a promoter operably associated with at least one gene encoding a control protein, wherein said control proteins interact with said promoters to maintain the state of said switch, and at least one gene provides a desired nucleic acid transcript or protein; and
(b) a gate structure that is capable of being in an on state or a off state; wherein a clocking agent substantially determines the state of the gate structure, and,
i. when the gate structure is in the on state, a multiplicity of switching agents can substantially determine the state of said genetic switch; and
ii. when the gate structure is in the off state, said switching agents have insubstantial effect on the state of said genetic switch;
wherein said clocked latch is resistant to switching agent fluctuation and eases the implementation of systems formed from multiple latches of similar design, and, the level of at least one desired nucleic acid transcript or protein is controlled.

2. The clocked latch of claim 1, wherein the gate structure comprises a stem and loop structure formed in a nucleic acid construct.

3. The clocked latch of claim 2, wherein the clocking agent is temperature.

4. The clocked latch of claim 2, wherein the clocking agent is an amino acid or its corresponding transfer-ribonucleic acid.

5. The clocked latch of claim 1, wherein the gate structure is a nucleic acid construct incorporating a termination site.

6. The clocked latch of claim 1, wherein the gate structure is a nucleic acid construct comprising a terminator site, a spacer and a utilization site.

7. The clocked latch of claim 2, 5 or 6, wherein the clocking agent is a protein.

8. A host cell harboring the clocked latch of claim 3, 4, or 7.

9. The host cell of claim 8, wherein the host cell is a prokaryotic cell.

10. The host cell of claim 9, wherein the prokaryotic cell is Escherichia coli.

11. The host cell of claim 10, wherein the host cell is an eukaryotic cell.

12. A method of alternating transcription from first and second promoters in a host cell, the method comprising the steps of:
(i) providing a host cell harboring a recombinant genetic clocked latch comprising:
a) a bistable genetic switch that is capable of being substantially stable in a first state or in a second state, the switch comprising nucleic acid constructs, each construct comprising a promoter operably associated with at least one gene encoding a control protein, wherein said control proteins interact with said promoters to maintain the state of said switch, and at least one gene provides a desired nucleic acid transcript or protein, where:
1. in the first state, transcription is substantially from the first promoter and acts to repress transcription from the second promoter; and
2. in the second state, transcription is substantially from the second promoter and acts to repress transcription from the first promoter; and
b) a gate structure that is capable of being in an on state or a off state;
wherein a clocking agent substantially determines the state of the gate structure, and,
1. when the gate structure is in the on state, switching agents can substantially determine the state of said genetic switch; and
2. when the gate structure is in the off state, said switching agents have insubstantial effect on the state of said genetic switch; and
(ii) providing the clocking agent and either:
a) the first switching agent to derepress transcription of the first gene by the first promoter and act to repress transcription from the second promoter;
b) or the second switching agent to derepress transcription of the second gene by the second promoter and act to repress transcription from the first promoter,
wherein transcription does not substantially alternate between said first and second promoters in the absence of said clocking agent, and, the level of at least one desired nucleic acid transcript or protein is controlled.

13. The method of claim 12, wherein the gate structure comprises a stem and loop structure formed in a nucleic acid construct.

14. The method of claim 13, wherein the clocking agent is temperature.

15. The method of claim 13, wherein the clocking agent is an amino acid or its corresponding transfer-ribonucleic acid.

16. The method of claim 12, wherein the gate structure is a nucleic acid construct incorporating a termination site.

17. The method of claim 17, wherein the clocking agent is a protein.

18. The method of claim 12, wherein the gate structure is a nucleic acid construct comprising a terminator site, a spacer and a utilization site.

19. The method of claim 13, 16 or 18 wherein the clocking agent is a protein.

20. The method of claim 13, wherein the host cell is a prokaryotic cell.

21. The method of claim 18, wherein the prokaryotic cell is Escherichia coli.

22. The method of claim 13, wherein the host cell is an eukaryotic cell.


SEQUENCE LISTING (10 sequences)

SEQ ID NO: 1 - DNA, 36 bp, Artificial
  SWITCHfwd primer for clocked latch genetic circuit
gcgcgcgcggatcccttgccgatcagccaaacgtct

SEQ ID NO: 2 - DNA, 36 bp, Artificial
  SWITCHrev primer for clocked latch genetic circuit
cgcgcgcgaattcggctggaatgtgtaagagcgggg

SEQ ID NO: 3 - DNA, 87 bp, Artificial
  TermPos tL3 terminator and N-utilization site from phage lambda
tcgagcacattccagccctgaaaaagggcatcaaattacaaagcgaggccgggtatttcccggcctttctgttatccgaaatccacg

SEQ ID NO: 4 - DNA, 87 bp, Artificial
  TermNeg tL3 termination and N-utilization site from phage lambda
aattcgtggatttcggataacagaaaggccgggaaatacccggcctcgctttgtaatttgatgccctttttcagggctggaatgtgc

SEQ ID NO: 5 - DNA, 43 bp, Artificial
  GFPaav fwd primer for clocked latch genetic circuit
gcgcgcggatccaggaaacagctatgcgtaaaggagaagaact

SEQ ID NO: 6 - DNA, 36 bp, Artificial
  GFPaav rev primer for clocked latch genetic circuit
cgcggcggccgcttaaactgctgcagcgtagttttc

SEQ ID NO: 7 - DNA, 67 bp, Artificial
  Term2Pos tL3 and N-utilization sites of phage lambda
ccattccagccctgaaaaagggcatcaattacaaagcgaggccgggtatttcccggcctttctgtgc

SEQ ID NO: 8 - DNA, 75 bp, Artificial
  Term2Neg tL3 and N-utilization sites of phage lambda
ggccgcacagaaaggccgggaaatacccggcctcgctttgtaattgatgccctttttcagggctggaatggagct

SEQ ID NO: 9 - DNA, 100 bp, Artificial
  Latchfwd primer for clocked latch genetic circuit
gcgcatcgatccctatcagtgatagagattgacatccctatcagtgatagagatactgagcacataaataaccccgctcttacgggccccccctcgagca

SEQ ID NO: 10 - DNA, 102 bp, Artificial
  Latchrev primer for construction of clocked latch
gacccgggatgtgagcggataacattgacattgtgagcggataacaagatactgagcacataaataaccccgctcttatagggcgaattggagctccattcc